(19) United States
(12) Patent Application Publication
(10) Pub. No.: US 2008/0174733 A1
(43) Pub. Date: Jul. 24, 2008

(54) Method of using infrared thermal imager to diagnose eye diseases and the device thereof

(75) Inventors: David O. Chang, Taipei Hsien (TW); Kenny Hui-Hua Chiang, Taipei City (TW); Ko-Hua Chen, Taipei (TW)

Correspondence Address: KAMRATH & ASSOCIATES P.A., 4825 OLSON MEMORIAL HIGHWAY, SUITE 245, GOLDEN VALLEY, MN, 55422, US

(73) Assignee: UNITED INTEGRATED SERVICES CO., LTD., Taipei City (TW)

(21) Appl. No.: 11/655,023

(22) Filed: Jan. 18, 2007

(51) Int. Cl. A61B 3/10

(52) U.S. Cl. 351/206

(57) ABSTRACT

A method of using infrared thermal imager to diagnose eye diseases including the dry eye syndromeuses an infrared thermal imager to take the surface image of the eyeball of the patient. Temperature variation at a specific position of the cornea of the eyeball is precisely measured in a unit time. An attenuation coefficient K of a temperature dropping curve and a temperature difference ΔT are obtained from the temperature variation. Optimized curves of K and ΔT are provided for the doctor to diagnose the patient's eye disease.

### BACKGROUND OF THE INVENTION

1. Field of the Invention

The invention relates to a method of using an infrared thermal imager to diagnose eye diseases and the device thereof. In particular, the invention relates to the technique of using an infrared thermal imager to take the image of the patient's eye and obtain the surface temperature variation of the eye to diagnose the eye diseases (including the dry eye syndrome).

2. Description of Related Art

The human eye has a tear layer protecting the surface of the cornea. The tear layer includes from inside out a mucus layer, an aqueous layer, and a lipid layer. The lipid layer on the outer surface of the eye is formed by the tarsal gland. The aqueous layer in the middle is produced by the lachrymal and accessory lachrymal glands. The inner mucus layer is produced by the conjunctival goblet cells. The functions of the three layers are complementary to one another. The lipid layer prevents the evaporation of water. The mucus layer evenly distributes tears over the surface of cornea. When a normal person blinks his/her eyes, the tear layer in front of the cornea is refreshed. The tear leaves the punctum via the canaliculus and reaches the nasal cavity. This mechanism makes the person feel comfortable in the eyes and clear in the eyesight. However, for a patient with the dry eye syndrome, the secretion and replacement of tears are worse. Therefore, they feel more uncomfortable.

As the tear secretion normally reduces with age, dry eye syndrome usually occurs to senior people. Nevertheless, there are more young patients suffering from the dry eye syndrome because of the worse living environment nowadays. In fact, the dry eye syndrome has become one of the most commonly seen diseases in the clinics (about 20%).

Usually, the dry eye syndrome requires some tests in order to correctly identify the disease. Such tests include:

1. The Schirmer test (basic “I” and “II” tests). It is used to test the basic and reflective secretion of tears.

2. The tear break up time (BUT) test. It is used to test the secretion quality.

In the Schirmer test, the eye is dropped with anesthetizing drugs for three times. Afterwards, a piece of long filter paper is disposed at the lower eyelid. One then measured the length of the wetted part of the filter paper after a few minutes. If it is less than 5 mm, then the person is diagnosed with the dry eye syndrome. As to the tear BUT test, the patient's eyes are dropped with a fluorescent dye. The time of the first break up after the patient opens his/her eyes is measured using a slit lamp. The person is diagnosed with the dry eye syndrome if the time is less than 10 seconds.

Although the above tests are often used to identify the dry eye syndrome, they have some disadvantages or inconvenience. For example, placing the filter paper at the lower eyelid has certain invasiveness to the eyes. Also, the patient may have fear with such a method. The test takes tens of minutes, causing discomfort to the patient. In the tear BUT test, the patient has to be dropped with a dye. This causes discomfort too and requires the corporation of the patient as well. In addition to fear and discomfort, the above-mentioned two methods have the drawbacks of low repeatability and high variability.

### SUMMARY OF THE INVENTION

The invasiveness in the traditional tests for diagnosing the dry eye syndrome is likely to cause fear and discomfort to the patient. There are also the drawbacks of long testing time, low repeatability, and high variability.

In view of the foregoing, an objective of the invention is to provide a non-invasive, high-repeatable, and low-variable test for diagnosing eye diseases. During the test process, the patient does not experience the fear and discomfort caused by the invasion of filter paper or dye to the eyes. The disclosed method has a high precision for the doctor to readily diagnose the dry eye syndrome.

To achieve the above-mentioned objective, the primary technique of the method involves the following steps:

taking the image of the surface of the patient's cornea with an infrared thermal imager and defining at least one temperature measuring point;

taking a series of images of the cornea surface within a unit time and obtaining the temperature variation at the temperature measuring point;

generating a temperature dropping curve according to the above temperature variation data, and obtaining an attenuation coefficient K;

computing the temperature difference ΔT at the temperature measuring point within the unit time; and

using the attenuation coefficient K and the temperature difference ΔT obtained in the previous steps as the basis for diagnosing the dry eye syndrome.

For normal people, new tear layers are produced after each blink to protect the corneas. However, patients with the dry eye syndrome have abnormal tear layers, resulting in a different surface temperature on the cornea. According to the literature, patients with the dry eye syndrome have a slower temperature in the cornea than normal people. It is probably because the quality of tear layer is so poor that there is no appropriate heat dissipation. Therefore, the attenuation coefficient K of the dry-eye-syndrome patient obtained from the above-mentioned method is lower than a normal person. Moreover, the temperature difference between the highest and lowest temperatures after the patient opens his/her eyes is smaller than a normal person. As a result, the attenuation coefficient K of the temperature dropping curve and the temperature difference ΔT between the highest and lowest temperatures can be used as reference for the doctor to diagnose the dry eye syndrome. It should be mentioned that the disclosed method uses an infrared thermal imager to take the pictures of the cornea surface of the patient. It is thus a non-invasive test. Not only does it prevent the patient from fear and discomfort, it also has the advantages of high repeatability and low variability.

The infrared thermal imager defines at least one temperature measuring point on the surface of the patient's cornea, particularly the center and symmetric edges of the cornea.

The temperature measuring points are the center of the cornea and four points symmetrically distributed on both sides of the center (two on each side), and all fall within the area of pupil.

The temperatures obtained from the center and other temperature measuring points are computed to obtain the variability among them. This information can be used to diagnose the seriousness of the dry eye syndrome.

Another objective of the invention is to provide an infrared eye disease diagnosing device. The device includes a fixture, an infrared thermal imager, a converting unit, and a computer system.

The fixture is provided with a forehead rest and a lower jaw support. They are used to position the patient's head during the test.

The infrared thermal imager is opposite to the fixture to take images of the surface of the patient's cornea. It further extracts the temperatures at specific positions of its surface for computing the variations thereof.

The converting unit is connected to the output terminal of the infrared thermal imager.

The computer system is built in with an operation process. Using the converting unit and the infrared thermal imager, it generates such data as temperature dropping curves and temperature differences from the temperatures obtained for specific positions of the patient's cornea.

The converting unit comprises a pre-amplifier, an analog-to-digital converter (ADC), and a dynamical image processor. The pre-amplifier pre-amplifies a thermal image signal. The ADC converts the analog thermal image signals into digital signals, which are then sent to the dynamical image processor for processing. Finally, the signals are sent to the computer system to compute the attenuation coefficient of the temperature dropping curve and the temperature difference.

The infrared thermal imager is installed with an adjusting mechanism that makes vertical and horizontal displacements for an optimized angle to take the thermal image of the surface of the patient's cornea.

The above-mentioned device further includes: a visible-light camera for simultaneously taking the image of the surface of the patient's cornea; a display connected to the visible-light camera for playing the captured cornea image; and a reflector disposed between the eye side and the display. The cornea image shown on the display is projected onto the reflector. The patient can watch the image on the reflector using his/her residual sight to adjust the eye position for the infrared thermal imager to accurately take the thermal image of the cornea.

### BRIEF DESCRIPTION OF THE DRAWINGS

FIG. 1 is a schematic view of using an infrared thermal imager to diagnose eye diseases in accordance with the present invention;

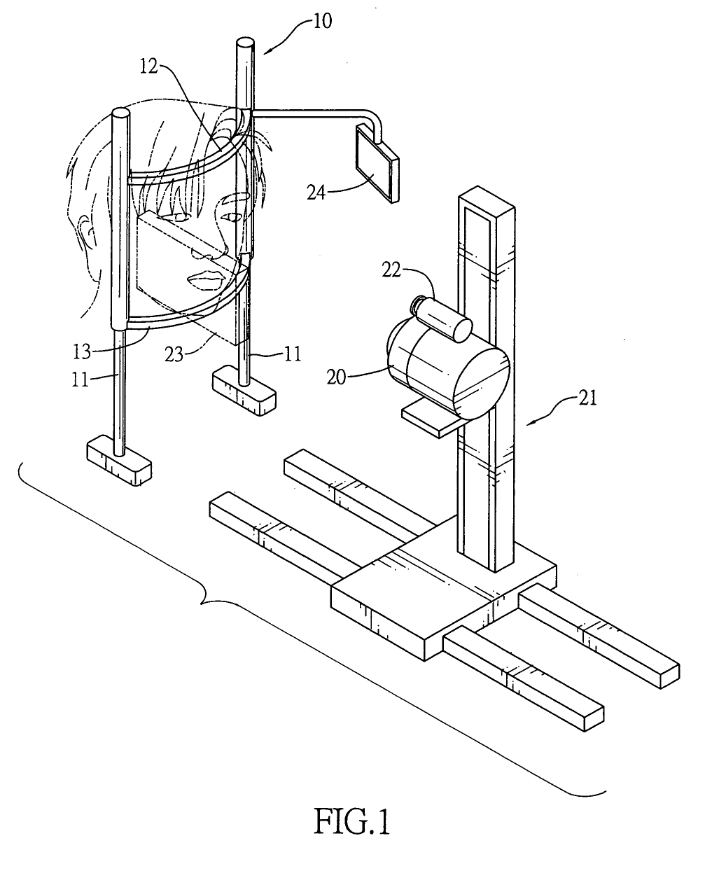

FIG. 2 is a planar diagram of using an infrared thermal imager to diagnose eye diseases in accordance with the present invention;

FIG. 3 is a circuit block diagram of the converting unit of the invention;

FIG. 4 is a schematic view of a cornea; and

FIG. 5 shows the relation between a cornea temperature and an open time of the eye.

### DETAILED DESCRIPTION OF THE PREFERRED EMBODIMENT

A system according to an embodiment of the invention is shown in FIG. The system includes a fixture 10 for a patient to rest his/her head, an infrared thermal imager 20, and a converting unit (not shown in the drawing). The infrared thermal imager 20 is connected to a computer system 40 (not shown in the drawing) via the converting unit.

The fixture 10 is provided with a forehead rest 12 and a lower jaw support 13 on the upper and lower ends of two parallel bars 11, respectively, for the patient to rest his/her head during the test. The patient puts his/her lower jaw on the lower jaw support 13 and his/her head against the forehead rest 12. In addition to fixing the head, the fixture also fixes the eyesight to a specific direction.

The infrared thermal imager 20 can be a focal plane array (FPA). It is installed opposite to the fixture 10 for taking the thermal images of the surface of the patient's cornea. In this embodiment, the infrared thermal imager 20 is mounted on an adjusting mechanism 21 for adjusting the height, angle and distance of the infrared thermal imager 20 with respect to the fixture 10. This allows the infrared thermal imager 20 to accurately take the thermal image of the cornea surface.

In front of the fixture and also in front of the patient's eyesight is provided with a fixed target (not shown). It is provided for the patient to watch directly for calibrating the image taking angle of the infrared thermal imager 20. The fixed target may be a low luminous light source or fluorescent source that does not make the patient's eye uncomfortable. The fixed target can be disposed at a surrounding position of the infrared thermal imager 20.

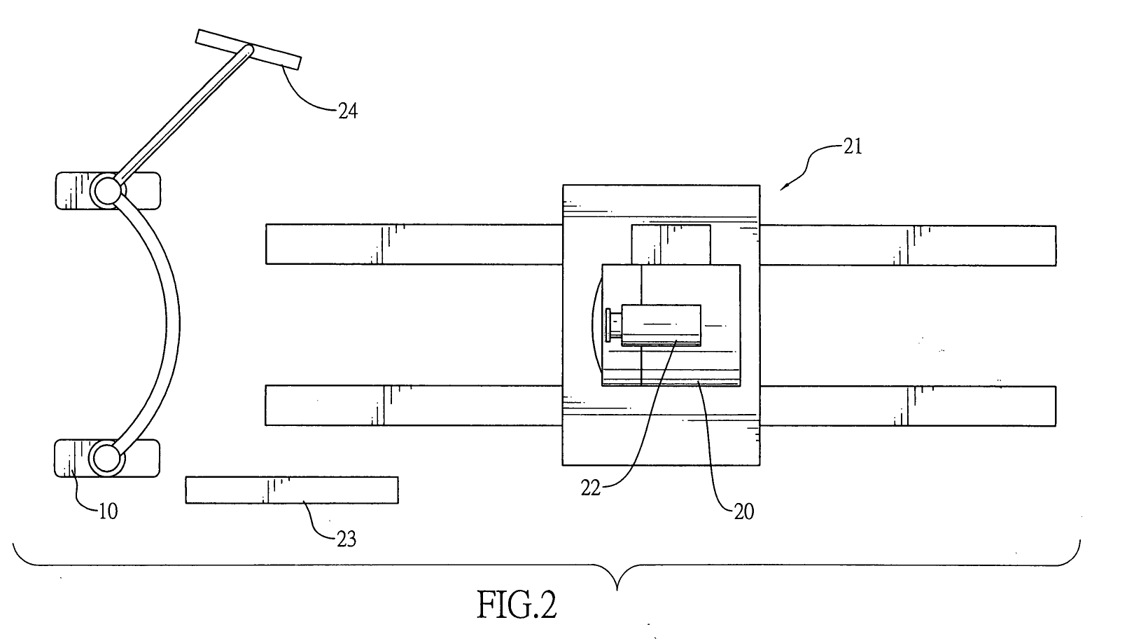

With reference to FIG. 2, the disclosed system further includes: a visible light camera 22, a display 23, and a reflector 24.

The visible light camera 22 is mounted on the infrared thermal imager for taking the visible light image of the surface of the patient's cornea. The display 23 is connected to the visible light camera 22 for showing the cornea image taken by the visible light camera 22. The reflector 24 is mounted on a side position in front of the fixture 10, between the position reachable by the patient's residual sight and the display. The cornea image shown on the display 23 can be projected onto the reflector 24, so that the patient can see his/her own eye in the reflector 24 using the residual sight for adjusting the position of eye. The infrared thermal imager 20 can therefore accurately take the thermal image of the cornea.

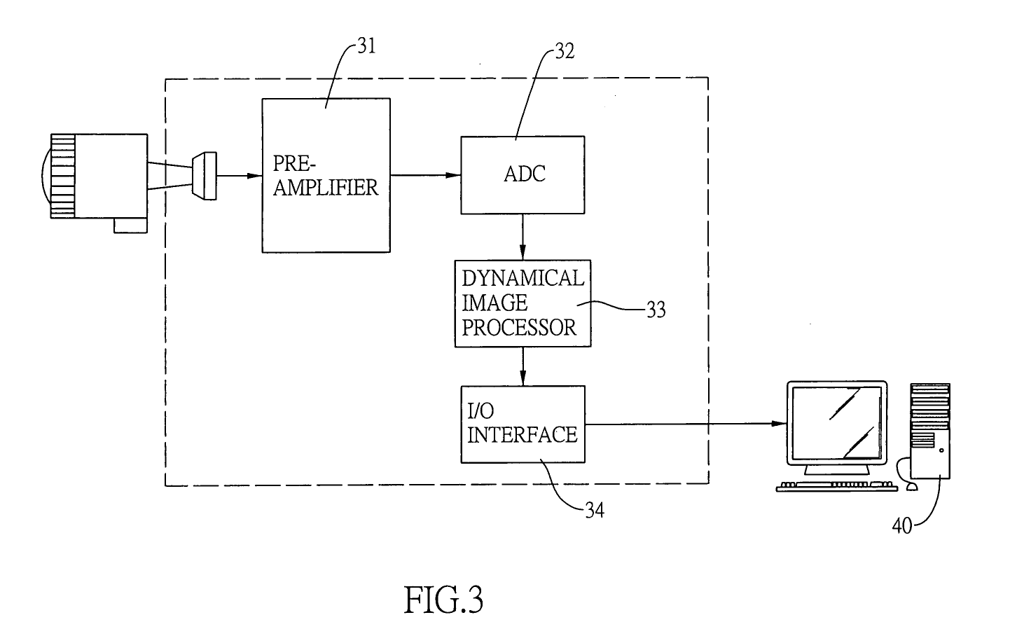

The structure of the converting unit is shown in FIG. 3 and includes a pre-amplifier 31, an analog-to-digital converter (ADC) 32, a dynamical image processor 33, and an input/output (I/O) interface 34.

The input terminal of the pre-amplifier 31 is connected to the output terminal of the infrared thermal imager 20 for pre-amplifying the thermal image signals output by the infrared thermal imager 20. The ADC 20 converts the originally analog and pre-amplified thermal image signals into digital signals. The dynamical image processor 33 is a processor that eliminates image noises to produce smooth images. The produced images with better quality are sent to the computer system 40. The I/O interface 34 is disposed between the dynamical image processor 33 and the computer system 40 for transmitting the digital thermal image signals to the computer system 40.

The computer system 40 is built in with a control operation procedure. According to the temperatures obtained at specific positions of the patient's cornea by the infrared thermal imager 20, the control operation procedure generates such data as a temperature dropping curve and a temperature difference. The procedure and test method of the control operation procedure are described as follows.

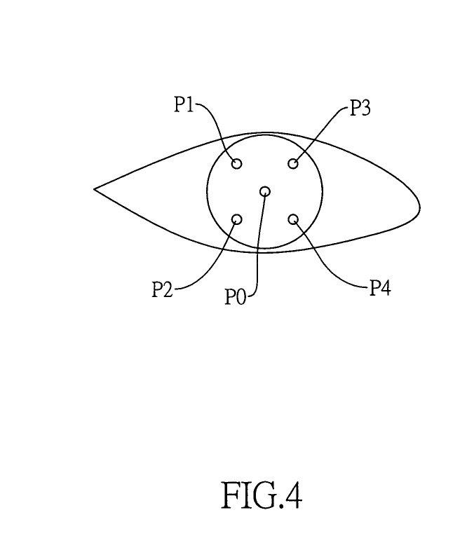

FIG. 4 shows a cornea. As described above, the invention uses the infrared thermal imager 20 to take the thermal image of the patient's cornea surface. The control operation procedure in the computer system 40 defines at least one temperature measuring point on the patient's cornea. In this embodiment, the cornea center and four points at equal distance from the center are defined as five temperature measuring points P0˜P4. They are all within the range of the pupil. The control operation procedure of the computer system 40 uses the infrared thermal imager 20 to extract temperature information within a unit time. The temperature extraction procedure involves the following steps:

1. The examinee is asked to close his/her eyes. The temperature extraction procedure is set to take 12 seconds.

2. Once the procedure starts, TRA temperature calibration is performed in the first second. A prompting beep is generated at the second, prompting the examinee to open his/her eyes and watch a fixed target in the front.

3. Temperatures at the five temperature measuring points P0˜P4 on the cornea are extracted periodically (e.g., 12 times each second) and stored.

4. When the procedure is over, a prompting beep is generated to notify the examinee.

After the above-mentioned extraction procedure is over, the computer system 40 stores several sets of temperature data. These data are used to generate the following information:

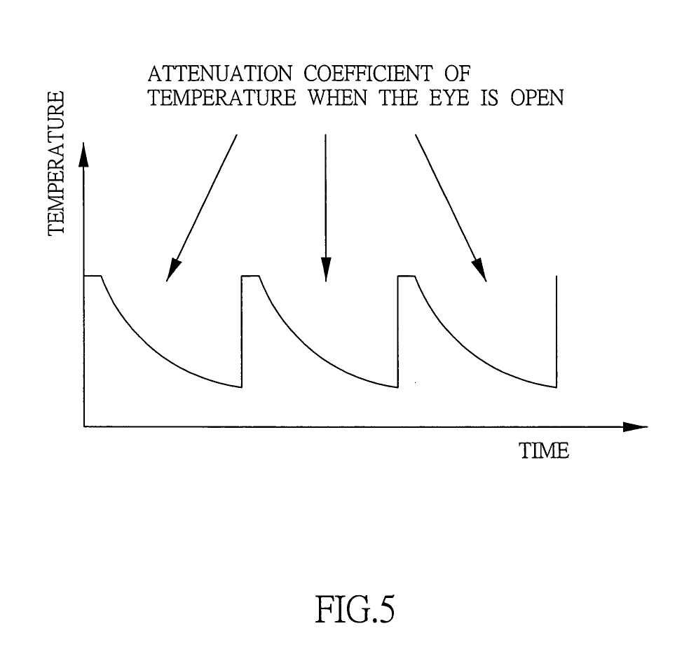

1. The temperature dropping curve for the cornea center P0 (FIG. 5). The highest point of the curve is the temperature at the cornea center when the examinee just opens his/her eye. The temperature decreases as heat dissipates. The lowering slope K of the curve is defined as an attenuation coefficient.

2. The temperature difference ΔT between the highest and lowest temperatures at the cornea center P0.

3. The temperature variation VA between the cornea center P0 and the other four temperature measuring points P1˜P4.

The attenuation coefficient K of the dry eye syndrome patient is lower than that of a normal person. The temperature difference between the highest and lowest temperatures of the patient is also smaller than that of a normal person. Therefore, the analysis of the K value and the temperature difference can be used to help diagnosing the dry eye syndrome.

Although we have used the dry eye syndrome as an example in the previous embodiment, the invention can be used to diagnose other diseases as well. In fact, many eye problems actually result from other diseases. For example, the commonly seen diseases such as diabetes, hypertension, and thyroid gland disease can be diagnosed from the pathological or color changes in the eye. By computing and analyzing the surface temperature and color of the eye along with other related parameters, the invention can help doctors diagnosing the above-mentioned diseases.

In comparison with the traditional Schirmer test and tear BUT test, the invention for diagnosing eye diseases, particularly the dry eye syndrome, has the following differences and advantages:

1. It is a non-invasive test. It prevents the patient from the discomfort and fear caused by filter paper and/or dye. It further reduces the possibility of infection.

2. It provides a reliable, quantitative test data to avoid human errors from simple observations.

What is claimed is:

1. A method of using an infrared thermal imager to diagnose eye diseases, comprising the steps of:
using the infrared thermal imager to take images of a surface of a cornea and defining at least one temperature measuring point;
continuously capturing the images of the surface of the cornea within a unit time and obtaining temperature variation data at the temperature measuring point;
generating a temperature dropping curve according to the temperature variation data and obtaining an attenuation coefficient K; and
computing a temperature difference ΔT within the unit time at the temperature measuring point according to the temperature variation data.

2. The method as claimed in claim 1, wherein the infrared thermal imager defines multiple temperature measuring points on the surface of cornea, including the cornea center and points at equal distance from the center.

3. The method as claimed in claim 2, wherein the temperature measuring points comprise the cornea center and four points at equal interval on both sides, two on each side, and within the range of a pupil.

4. The method as claimed in claim 3, wherein the temperature variation data measured at the cornea center and other temperature measuring points are used to compute the variations among them for diagnosing the seriousness of the eye disease.

5. The method as claimed in claim 1 being suitable for diagnosing the dry eye syndrome.

6. The method as claimed in claim 2 being suitable for diagnosing the dry eye syndrome.

7. The method as claimed in claim 3 being suitable for diagnosing the dry eye syndrome.

8. The method as claimed in claim 4 being suitable for diagnosing the dry eye syndrome.

9. A device of using an infrared thermal imager to diagnose eye diseases, comprising:
a fixture for positioning the head of a patient;
an infrared thermal imager disposed opposite to the fixture for taking thermal images of the patient's cornea surface to generate thermal image signals and obtaining temperatures at specific position on the cornea surface;
a converting unit connected to the output terminal of the infrared thermal imager; and
a computer system connected to the infrared thermal imager via the converting unit and built in with the method of claim 1 for obtaining a temperature dropping curve and temperature differences according to the temperatures extracted from specific positions on the patient's cornea surface.

10. The device as claimed in claim 9, the converting unit comprising:
a pre-amplifier to amplify the thermal image signals;
an analog-to-digital converter (ADC) to convert the thermal image signals into a digital signal; and
a dynamical image processor to receive the digital signal, eliminate image noises of the digital signal and transmit the digital signal to the computer system for obtaining the temperature dropping curve and the temperature differences.

11. The device as claimed in claim 9, wherein the infrared thermal imager is mounted on an adjusting mechanism that makes vertical and horizontal displacements to reach an appropriate angle for taking the thermal images of the patient's cornea surface.

12. The device as claimed in claim 9 further comprising:
a visible light camera for simultaneously taking images of the patient's cornea surface;
a display connected to the visible light camera for showing the cornea images captured thereby; and
a reflector disposed between a position reachable by the patient's residual sight and the display, wherein the cornea image shown on the display is projected onto the reflector so that the patient can adjust his/her eye position for the infrared thermal imager to accurately obtain the thermal image of the cornea.

13. The device as claimed in claim 10 further comprising:
a visible light camera for simultaneously taking images of the patient's cornea surface;
a display connected to the visible light camera for showing the cornea images captured thereby; and
a reflector disposed between a position reachable by the patient's residual sight and the display, wherein the cornea image shown on the display is projected onto the reflector so that the patient can adjust his/her eye position for the infrared thermal imager to accurately obtain the thermal image of the cornea.

14. The device as claimed in claim 11 further comprising:
a visible light camera for simultaneously taking images of the patient's cornea surface;
a display connected to the visible light camera for showing the cornea images captured thereby; and
a reflector disposed between a position reachable by the patient's residual sight and the display, wherein the cornea image shown on the display is projected onto the reflector so that the patient can adjust his/her eye position for the infrared thermal imager to accurately obtain the thermal image of the cornea.

15. The device as claimed in claim 12, wherein a fixed target is provided in front of the fixture for the patient to look at.

16. The device as claimed in claim 13, wherein a fixed target is provided in front of the fixture for the patient to look at.

17. The device as claimed in claim 14, wherein a fixed target is provided in front of the fixture for the patient to look at.

18. The device as claimed in claim 15, wherein the fixed target is disposed at a position beside the infrared thermal imager.

19. The device as claimed in claim 16, wherein the fixed target is disposed at a position beside the infrared thermal imager.

20. The device as claimed in claim 17, wherein the fixed target is disposed at a position beside the infrared thermal imager.
